(19) United States
(12) Patent Application Publication
(10) Pub. No.: US 2020/0279638 A1
(43) Pub. Date: Sep. 3, 2020

(54) MEDICAL IMAGE DISPLAY DEVICE AND TRACE LINE PROCESSING METHOD

(71) Applicant: Hitachi, Ltd., Tokyo (JP)

(72) Inventor: Suguru Ishiguro, Tokyo (JP)

(73) Assignee: Hitachi, Ltd., Tokyo (JP)

(21) Appl. No.: 16/646,689

(22) PCT Filed: Nov. 30, 2018
(86) PCT No.: PCT/JP2018/044247

§ 371 Date: Mar. 12, 2020

(30) Foreign Application Priority Data
Feb. 26, 2018 (JP) 2018-031709

(51) Int. Cl. G16H 30/40; G06T 11/20; G06F 16/54; A61B 8/00; A61B 8/14; A61B 8/08

(52) U.S. Cl. CPC G16H 30/40; G06T 11/203; G06F 16/54; G06T 2210/41; A61B 8/14; A61B 8/488; A61B 8/465

(57) ABSTRACT

A trace line is created by manually moving a pointer on an ultrasonic image. When the pointer is traced back along the trace line from a head coordinate, this operation is automatically determined to be a deletion operation and the trace line is partially deleted according to the amount of pointer movement during the operation. In determining the type of operation, the angle formed between the movement vector and a reference vector is compared to an angle threshold.

### TECHNICAL FIELD

The present invention relates to a medical image display device and a trace line processing method, and particularly, to technology of processing and displaying a trace line created manually.

### BACKGROUND

A medical image display device is a device configured to display a medical image such as an ultrasonic image, an X-ray CT image, or the like. The concept of the medical image display device includes a diagnostic device such as an ultrasonograph, an X-ray CT device, and the like, and an information processing device configured to display the medical image. The concept may also include a treatment device. Hereinbelow, the ultrasonograph is described as the medical image display device.

With the ultrasonograph, measurement is executed on an ultrasonic image, as required. For example, an area of a left ventricle of a heart is obtained. In this case, a contour of the left ventricle is manually traced. Specifically, when a track ball is operated on a tomographic image in a freeze state so as to move a pointer (cursor) along the contour of the left ventricle, a trace line simulating the contour of the left ventricle is generated. A start point and an end point of the trace line can be made to coincide with each other. However, when manually tracing the contour of the left ventricle, the start point and the end point are usually set on two annuli of heart valve and are linearly connected. In some cases, a contour of a tissue other than the heart may be manually traced.

Patent Document 1 discloses technology of deleting a manually drawn curve point by point from a head thereof on a screen of an X-ray CT device. According to the technology, the curve is deleted by an input signal separate from an input signal for drawing the curve. Patent Document 2 discloses technology of, when a box-shaped cursor is set on a curve drawn on a medical image, automatically determining whether or not to delete a curved portion of the curve, based on a length of the curved portion.

### CITATION LIST

### Patent Literature

Patent Document 1: JP 61-8433 B

Patent Document 2: JP 2013-255664 A

### SUMMARY

### Technical Problem

While manually tracing a target object on the medical image, it may be intended to delete the trace line within a predetermined range from a head of the trace line. In this case, when a special deletion operation such as a knob rotating operation is required, it is necessary to detach a hand from a pointing device that is being operated by the hand, and to perform the deletion operation with the hand. Alternatively, it is necessary to perform the deletion operation with a hand separate from the hand operating the pointing device. Such a deletion operation is troublesome for a user. Also, when performing the deletion operation, it is necessary to move the line of sight from the medical image displayed on the screen to an operation panel, which degrades operation efficiency of the manual tracing. Thus, it is necessary to conveniently delete the trace line from the head thereof by using the pointing device, as it is, which has been operated up to that time.

An object of the present invention is to improve operability when deleting partially or wholly a current trace line drawn on a medical image. Also, an object of the present invention is to enable a deletion operation to be performed with a similar operation feeling to that of a tracing operation by a pointing device.

### Solution to Problem

A medical image display device disclosed herein includes a device for drawing a trace line by moving a pointer on a medical image, a determination unit configured to determine, based on a current trace line extending from a start point coordinate to a current head coordinate and a current pointer coordinate after the current head coordinate is set, whether a pointer moving operation from the current head coordinate to the current pointer coordinate is a tracing operation or a deletion operation, and a deletion unit that deletes partially or wholly the current trace line when the deletion operation is determined.

A trace line processing method disclosed herein includes a process of determining, based on a current trace line extending from a start point coordinate to a current head coordinate and a current pointer coordinate after the current head coordinate is set, whether a pointer moving operation from the current head coordinate to the current pointer coordinate is a tracing operation or a deletion operation, a process of updating the current head coordinate to the current pointer coordinate when the tracing operation is determined, and a process of deleting partially or wholly the current trace line when the deletion operation is determined.

In the above configuration, the “current trace line”, the “current head coordinate”, and the “current pointer coordinate” indicate a trace line, a head coordinate, and a pointer coordinate at any point of time, on the assumption that the trace line, the head coordinate, and the pointer coordinate are sequentially updated over time. In descriptions below, when there is no particular problem in understanding of an embodiment even though a temporal relation is not explicitly described or when the temporal relation is generally expressed in an abstract manner, they will be simply referred to as “trace line”, “head coordinate”, and “pointer coordinate”, respectively.

### BRIEF DESCRIPTION OF DRAWINGS

FIG. 1 is a block diagram depicting an ultrasonograph according to an embodiment.

FIG. 2 depicts a drawing of a trace line based on a plurality of trace point coordinates.

FIG. 3 depicts an example of a management table.

FIG. 4 depicts a trace line drawn along a left ventricle contour.

FIG. 5 depicts the trace line having a portion deviating from the left ventricle contour.

FIG. 6 depicts partial deletion of the trace line.

FIG. 7 is a flowchart showing control contents of a trace line processing unit.

FIG. 8 depicts a pointer moving operation.

FIG. 9 depicts a relation between a movement vector and a reference vector.

FIG. 10 depicts another relation between the movement vector and the reference vector.

FIG. 11 depicts deletion operation determination.

FIG. 12 depicts tracing operation determination.

FIG. 13 depicts operation type determination based on a large angle threshold value.

FIG. 14 depicts deletion processing and generation of a new head coordinate.

FIG. 15 depicts a modified embodiment of the reference vector.

FIG. 16 depicts a modified embodiment of a deletion range.

FIG. 17 depicts another determination condition.

FIG. 18 depicts a first display example.

FIG. 19 depicts a second display example.

### DESCRIPTION OF EMBODIMENTS

Hereinbelow, an embodiment will be described with reference to the drawings.

### (1) Outline of Embodiment

A medical image display device of an embodiment includes a device, a determination unit, and a deletion unit. The device is a tool for drawing a trace line by moving a pointer on a medical image. The determination unit is configured to determine, based on a current trace line extending from a start point coordinate to a current head coordinate and a current pointer coordinate after the current head coordinate is set, whether a pointer moving operation from the current head coordinate to the current pointer coordinate is a tracing operation or a deletion operation. The deletion unit deletes partially or wholly the current trace line when the deletion operation is determined.

According to the above configuration, when the pointer is moved on the medical image by using the device, it is automatically determined whether the pointer moving operation is the tracing operation or the deletion operation. When it is determined that the pointer moving operation is a deletion operation, the current trace line is deleted partially or wholly. For example, the current trace line is deleted from a head thereof by a pointer moving amount. The remaining trace line after the deletion becomes a new current trace line. When the deletion operation is repeated, the trace line becomes gradually shorter.

The operation type is determined, based on the current trace line and the current pointer coordinate. In the deletion operation, the current trace line is a deletion target, and the current pointer coordinate designates the deletion target or a side on which the deletion target substantially exists. On the other hand, in the tracing operation, the current pointer coordinate sets a direction in which the trace line is made to extend from the current head coordinate. That is, it is possible to guess the operation type (user's intention) with reference to the current trace line and the current pointer coordinate. In other words, the tracing operation and the deletion operation are performed by the user on the premise of such rules.

In the embodiment, when it is intended to perform the deletion operation, it is preferable to move the pointer so that the deletion operation determination condition is to be satisfied. While performing the deletion operation, a special operation (for example, a click operation and a knob rotating operation) other than the pointer moving operation is not required. When the tracing operation is determined, the trace line is extended as a result of the pointer moving operation, like in the related art.

In the embodiment, the determination unit is configured to set a deletion operation determination condition, based on the current trace line, and determines the deletion operation when the deletion operation determination condition is satisfied by the pointer moving operation. The current trace line, particularly, a portion close to the head coordinate is a reference for estimating a direction or range in which a future tracing operation is highly likely to be performed, and at the same time, may also be a reference for determining the deletion operation. For example, it is determined that a “tracing operation” of returning the pointer along the current trace line is the deletion operation. Each part of a contour of a biological tissue is substantially rounded, and there is substantially no right-angle or V-shaped part in the contour. Thus, even if the above determination is made, it is unlikely that unintended deletion will be executed. In a special case, if the automatic determination about the operation type causes a decrease in operability, the automatic determination function about the operation type may be temporarily turned off.

In the embodiment, the determination unit is configured to set an angle condition as the deletion operation determination condition, based on a reference vector from the current head coordinate to a reference point coordinate on the current trace line, and determines the deletion operation when a movement vector from the current head coordinate to the current pointer coordinate satisfies the angle condition. That is, the angle condition is set based on the portion of the trace line close to the current head coordinate, and the operation type is determined based on whether the angle condition is satisfied by the pointer moving operation. The current pointer coordinate, the pointer moving direction, and the pointer moving amount are decided by the pointer moving operation. Among the information, the operation type is determined using one or more pieces of information. In the above configuration, the pointer moving direction of the information is used to determine the operation type. Also, the operation type may be determined in consideration of the pointer moving amount. Alternatively, the operation type may be determined, based on the current pointer coordinate.

In the embodiment, the determination unit is configured to change the deletion operation determination condition, based on at least one of a previous determination result and a pointer stop time. In general, in many cases the same operation is repeatedly executed. Therefore, the deletion operation determination condition is preferably set in consideration of the previous determination result. In the meantime, in a case in which the tracing operation is again performed after the deletion operation, usually, the pointer often stops between the operations. That is, when the pointer stop time reaches a predetermined level, a possibility that the tracing operation has resumed increases. In the above configuration, the deletion operation determination condition is adaptively set, considering at least one of the previous determination result and the pointer stop time, based on such an empirical rule.

In the embodiment, the reference point coordinate is a coordinate that is set by tracing back the current trace line from the current head coordinate, and a returning amount (tracing-back amount) at that time is set according to a pointer moving amount from the current head coordinate to the current pointer coordinate. When the pointer moving amount is large, the returning amount increases, and a reference coordinate representative of a large portion of the current trace line to be connected to the head coordinate is specified. When the pointer moving amount is small, the returning amount decreases, and a reference coordinate representative of a small portion of the current trace line to be connected to the head coordinate is specified. The returning amount may be set stepwise or continuously according to the pointer moving amount.

In the embodiment, the deletion unit is configured to set a deletion amount by which the current trace line is to be deleted with being traced back from the current head coordinate, based on the pointer moving amount from the current head coordinate to the current pointer coordinate. According to this configuration, since the deletion amount is changed by the pointer moving amount, the pointer moving amount is preferably set large (a pointer moving speed is preferably set fast) when it is intended to roughly delete the trace line, and the pointer moving amount is preferably set small (pointer moving speed is preferably set slow) when it is intended to finely delete the trace line. This is based on an idea of reflecting the user's intention in the deletion amount.

In the embodiment, the current trace line is managed as coordinate series composed of a plurality of trace point coordinates. When deleting partially the current trace line, some of the coordinate series are invalidated according to the deletion amount, and a new head coordinate is then added to a head of the remaining coordinate series. In this configuration, the current head coordinate is newly added to a point equivalent to the returning amount. The deletion processing may be executed in a trace point coordinate unit.

In the embodiment, whenever the current pointer coordinate is updated, it is determined whether the pointer moving operation is the deletion operation. When the pointer is continuously traced back along the current trace line, the current trace line becomes gradually shorter.

In the embodiment, the medical image display device includes a display for displaying at least one of a determination reference in the determination unit and a determination result of the determination unit. According to this configuration, it is possible to further improve the operability or to prevent or reduce an erroneous operation.

The trace line processing method of the embodiment is implemented as functions of hardware or software. In the latter case, a program configured to execute the method is installed in the medical image display device via a network or a portable storage medium.

### (2) Configuration of Embodiment

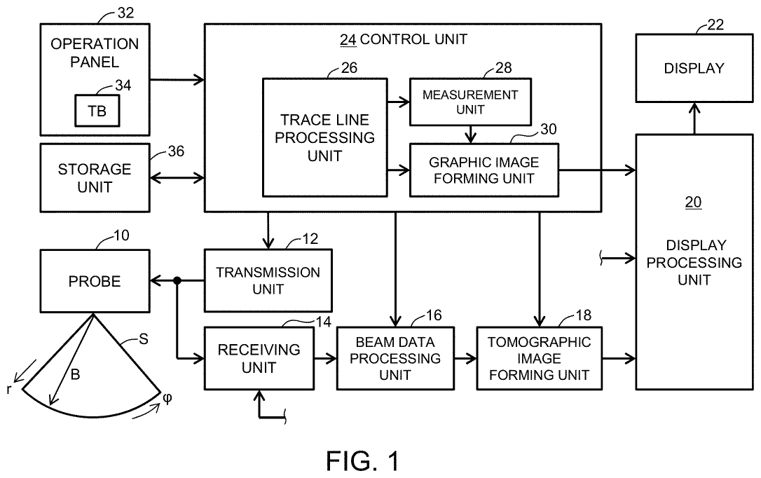

FIG. 1 depicts an ultrasonograph, as an example of the medical image display device. The ultrasonograph is equipped in a medical institution such as a hospital, and is configured to form an ultrasonic image, based on received data obtained by transmitting and receiving ultrasonic waves to and from a living body.

In the shown configuration example, a probe 10 is configured to transmit and receive ultrasonic waves in a state in which it is in contact with a surface of the living body. The probe 10 includes a vibration element array having a plurality of vibration elements arranged one-dimensionally. An ultrasonic beam B is formed by the vibration element array and is used for electronic scanning. Thereby, a beam scanning plane S is formed. As an electronic scanning method, a linear electronic scanning method and the like are known, in addition to the illustrated electronic sector scanning method. Instead of the vibration element array, a two-dimensional vibration element array may be provided. An intra-body cavity probe may also be used. In FIG. 1, an r direction is a depth direction, and a ϕ direction is a direction of electronic scanning with an ultrasonic beam.

A transmission unit 12 is an electronic circuit configured to function as a transmission beam former. Upon transmission, the transmission unit 12 is configured to output a plurality of transmission signals, which have been subjected to delay processing, to the vibration element array in parallel. Thereby, the ultrasonic waves are emitted into the living body. Upon reception, when reflected waves from an inside of the living body are received by the vibration element array, a plurality of reception signals are output from the vibration element array to a receiving unit 14.

The receiving unit 14 is an electronic circuit configured to function as a receiving beam former. Specifically, the receiving unit 14 is configured to apply phasing addition (delay addition) to the plurality of reception signals, thereby outputting beam data, as reception signals after the phasing addition. A plurality of beam data, which are aligned in the electronic scanning direction, are acquired every electronic scanning operation with the ultrasonic beam B, so that reception frame data is configured. Each beam data is configured by a plurality of echo data aligned in the depth direction. The receiving unit 14 includes a plurality of A/D converters and the like, which are not shown.

A beam data processing unit 16 is an electronic circuit configured to apply various types of signal processing such as wave detection, logarithmic conversion, and the like to each beam data. Each beam data after the processing is transmitted to a tomographic image forming unit 18. The tomographic image forming unit 18 is an electronic circuit configured to form a tomographic image (B mode image), based on the reception frame data, and includes a digital scan converter (DSC). The DSC is an electronic circuit having a coordinate conversion function and a pixel interpolation function. Data indicative of the tomographic image is transmitted to a display processing unit 20.

The display processing unit 20 has an image synthesis function, a color processing function, and the like. In the embodiment, the tomographic image and a graphic image are synthesized, so that a synthesis image is generated. Data indicative of the synthesis image is output to a display 22. The graphic image is a reference image or auxiliary image as a non-ultrasonic image including a character, a figure, and the like, and is displayed while being superimposed on the tomographic image. In the embodiment, the graphic image includes a trace line generated by manual tracing. Also, the graphic image includes a pointer (cursor) indicative of a coordinate currently instructed or designated by the user, a start point mark indicative of a start point of the trace line, and the like. The display is configured by an LCD, an organic EL display device, or the like. Meanwhile, a three-dimensional image formed by volume rendering, a color Doppler image indicative of a blood flow, an elasticity image indicative of elasticity information, and the like may also be displayed. The manual tracing may be executed on the ultrasonic images.

A control unit 24 is configured by a CPU and an operation program. The control unit 24 is configured to control operations of the respective units of the ultrasonograph. In FIG. 1, some main functions of the control unit 24 are shown by blocks. Specifically, a trace line processing unit 26, a measurement unit 28, and a graphic image forming unit 30 are shown. These functions may also be implemented by another processor or another device. The trace line processing unit 26 is a module configured to control or manage the manual tracing, and functions as a determination unit or a determination means and a deletion unit or a deletion means. The trace line processing unit 26 is configured to execute the trace line processing method of the embodiment.

The control unit 24 is connected with an operation panel 32. The operation panel 32 includes a track ball 34 as a pointing device, in addition to a plurality of switches, a plurality of knobs, and a keyboard. The track ball 34 has a freely rotatable ball, and is configured so that the ball is rotated by a finger or a palm, thereby instructing a pointer movement destination. Instead of the track ball 34, a mouse, a pen tablet, or another pointing device may also be used. In the embodiment, the track ball is subjected to a rotation operation, so that manual tracing of an internal organ contour is executed. For example, when the pointer is moved along a tissue contour on the tomographic image in a freeze state (still image state), a trace line is manually generated. The trace line may be manually generated on a moving image.

When the trace line deviates from the tissue contour, the trace line is partially deleted from a head thereof by a user's deletion operation. In a case in which a current trace line is relatively short, the trace line is wholly deleted from the head thereof, depending on content of the deletion operation. With the ultrasonograph of the embodiment, the trace line can be deleted by rotating a predetermined knob. In addition, the trace line can be naturally deleted by returning the pointer along the trace line (by performing a tracing operation in a return manner), without performing a special operation such as a click operation, a knob operation, and the like. That is, it is possible to perform the deletion operation with an operation feeling similar to that of the tracing operation.

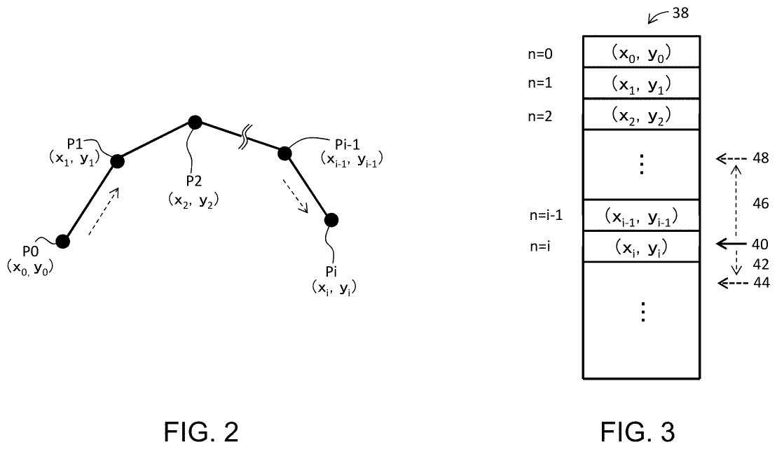

The trace line processing unit 26 is to execute processing for the manual tracing, and has, specifically, functions of managing (adding, deleting, updating, and the like) a plurality of trace point coordinates, drawing a trace line based on the plurality of trace point coordinates, determining an operation type (tracing operation and deletion operation), and the like. The respective functions are described in detail with reference to FIG. 2 and thereafter.

The measurement unit 28 is to execute designated measurement. For example, the measurement unit 28 is configured to calculate an area, based on a manual tracing result. In addition, the measurement unit has a variety of measurement functions such as distance measurement. The graphic image forming unit 30 is to generate a graphic image that is to be displayed with being superimposed on the tomographic image. The graphic image includes a trace line, a start point mark, an end point mark, and others (for example, information indicative of a status and information indicative of a determination condition). A storage unit 36 connected to the control unit 24 is configured by a semiconductor memory and the like, and a management table, which will be described later, is provided therein.

FIG. 2 depicts a trace line (current trace line) that is being made. The trace line is defined by a plurality of trace point coordinates. In the plurality of trace point coordinates, two adjacent trace point coordinates are connected by a demarcation line, in a pair unit of adjacent trace point coordinates, so that the trace line is configured as a connection of a plurality of demarcation lines. The trace line may be configured as a curve based on the plurality of trace point coordinates. For example, the trace line may be configured as a spline curve.

In FIG. 2, P0 indicates a start point coordinate. Pi indicates a current head coordinate. n indicates a coordinate number. The trace line is defined by (i+1) trace point coordinates from a number 0 to a number i. In the embodiment, a click operation or a switch operation is required only when setting the start point coordinate P0 and the end point coordinate. When deciding trace point coordinates other than the start point and end point coordinates, and during a deletion operation, which will be described later, an operation other than the track ball operation (pointer moving operation) is not required. In a screen, a horizontal direction is an x direction, and a vertical direction is a y direction. Each trace point coordinate is specified as an x coordinate and a y coordinate. A coordinate unit is a pixel.

FIG. 3 depicts an example of a management table for managing the plurality of trace point coordinates; i.e., the trace line. In the shown management table 38, (i+1) trace point coordinates ((i+1) coordinate data) from a trace point coordinate of a number 0 to a trace point coordinate of a number i are managed. Herein, the trace point coordinate of the number 0 is a start point coordinate, and the trace point coordinate of the number i is a current head coordinate. This is indicated by a reference sign 40. Next, when the tracing operation is performed, a trace point coordinate is added to a position indicated by a reference sign 44, which becomes a new head coordinate. On the other hand, when the deletion operation is performed, one or more trace point coordinates are partially deleted from the current head coordinate, which is a start point, by a part equivalent to a pointer moving amount (by the same length), as shown with a reference sign 46, and a new head coordinate is then added to the remaining trace point coordinate series, as shown with a reference sign 48. This will be described in detail later. The existing trace point coordinate may also be the head coordinate. When the deletion operation is repeated (when a situation in which the determination is not switched to the tracing operation determination continues), the trace line becomes gradually shorter toward the start point coordinate.

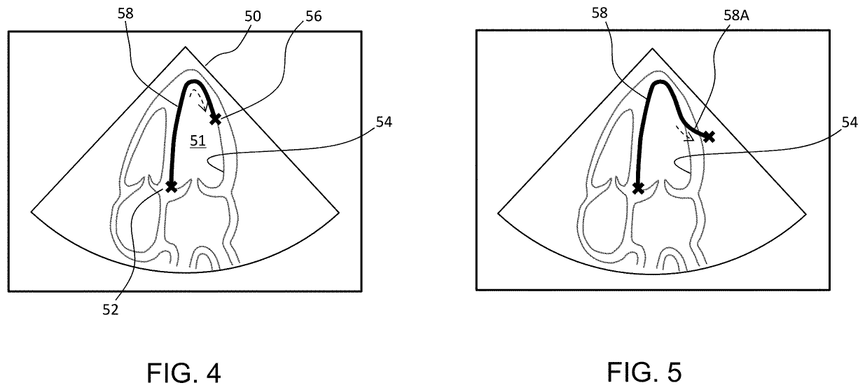

Specific examples of the tracing operation and the deletion operation are described with reference to FIGS. 4 to 6. FIGS. 4 to 6 depict a tomographic image of a heart.

In FIG. 4, a trace line 58 that is being made is drawn on a tomographic image 50. Specifically, the trace line 58 is drawn along a contour of a left ventricle 51, and extends from a start point coordinate to a current head coordinate. The start point coordinate is designated on one annulus of a heart valve of two annuli of the heart valve, which are both sides of a mitral valve. The end point coordinate is to be designated on the other annulus of heart valve. A start point mark 52 is indicated at the start point coordinate, and a pointer (cursor) 56 is indicated at the current head coordinate. The pointer is moved on the screen as a result of the rotation operation of the track ball. For example, the probe is held by a right hand, and the track ball is operated by a left hand.

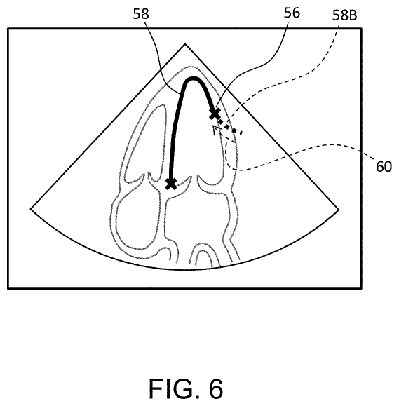

In FIG. 5, the trace line 58 deviates partially from a contour 54 of the left ventricle, so that a deviation portion 58A is generated. When it is intended to delete the deviation portion 58A and to again trace the corresponding portion, in the embodiment, it is preferable to perform an operation of returning the pointer along the trace line 58 from the current tip end coordinate (refer to a reference sign 60), as shown in FIG. 6. Alternatively, it is preferable to move the pointer from the current tip end coordinate toward the trace line 58 side. Although a deletion operation determination condition will be described in detail later, when the pointer moving operation is performed, the trace processing unit automatically determines that the operation is a deletion operation, not a tracing operation, and deletes a portion 58B of the trace line 58 from the head coordinate by the same amount as the pointer moving amount during the operation. On the other hand, when the pointer is moved from the current head coordinate so as to extend the current trace line, or when the pointer is moved forward, as seen from the current head coordinate, the trace processing unit automatically determines that the operation is a tracing operation, not a deletion operation, and sets the pointer movement destination coordinate as a new current head coordinate.

### (3) Operations of Embodiment

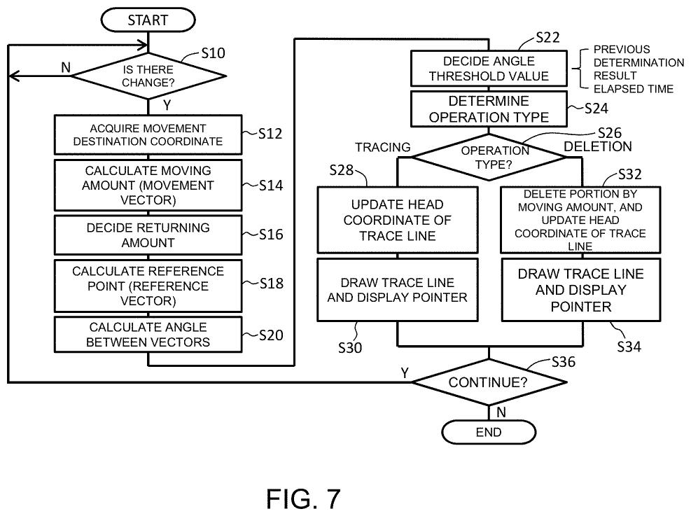

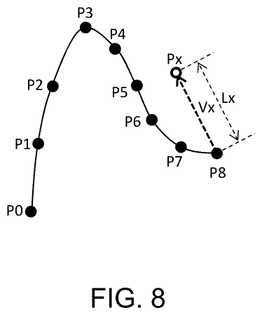

In each of FIG. 7 and thereafter, processing contents of the trace processing unit are more specifically shown. The processing contents of the trace processing unit are described in detail, based on FIG. 7, while referring to FIGS. 8 to 14. It should be noted that the processing contents shown in FIG. 7 are exemplary, and other processing contents may also be adopted inasmuch as it is possible to automatically determine the operation type as appropriate.

When an automatic determination function about the operation type becomes on, a series of processes shown in FIG. 7 are executed. In S10, it is determined whether there is a change in pointer coordinate. The pointer coordinate is measured at predetermined time intervals (for example, every 50 ms or 100 ms). When it is determined that there is a change in pointer coordinate, in S12, a movement destination coordinate of the pointer is acquired. In S14, a movement vector (a pointer moving direction and a pointer moving amount) facing from the head coordinate (current head coordinate) toward the movement destination coordinate (current movement destination coordinate) is calculated. The movement vector is described with reference to FIG. 8.

In FIG. 8, a trace line is generated, based on a plurality of trace point coordinates from P0 to P8. For example, the trace line is configured as a connection of a plurality of demarcation lines. P0 indicates a start point coordinate, and P8 indicates a current head coordinate. Px indicates the movement destination coordinate. A vector facing from the head coordinate P8 toward the movement destination coordinate Px is a movement vector Vx. In FIG. 8, the movement vector is slightly exaggerated. A moving amount of the pointer at that time is indicated by Lx.

In FIG. 7, in S16, a returning amount for setting a reference point coordinate, which will be described later, is decided, based on the moving amount Lx. For example, when the moving amount Lx is equal to or greater than a threshold value Lth, a large value L1 is decided as the returning amount, and when the moving amount Lx is less than the threshold value Lth, a small value L2 is decided as the returning amount. In the embodiment, the returning is switched in two steps. However, the returning amount may be switched in multiple steps or continuously. In S18, a reference point is specified as a point on the trace line, based on the returning amount. At the same time, a reference vector, which will be described later, is calculated. The reason to switch the returning amount is described, as follows. When it is intended to finely correct the trace line, it is expected that the moving amount Lx will decrease; i.e., it can be assumed that a vicinity of the current head coordinate is to be deleted. On the other hand, when it is intended to roughly correct the trace line, it is expected that the moving amount Lx will increase; i.e., it can be assumed that a large portion of the trace line continuing to the head coordinate is to be deleted.

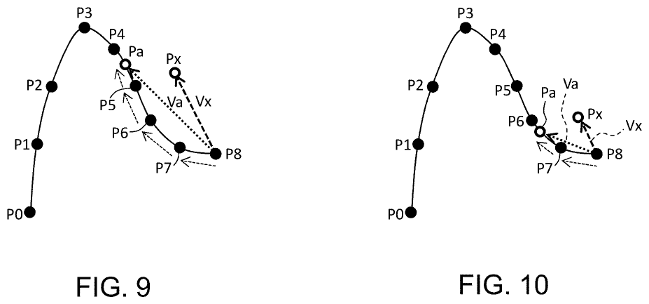

For example, when the large returning amount L1 is decided, a reference point coordinate Pa is set as a point traced back along the trace line from the head coordinate P8 by the returning amount L1, as shown in FIG. 9. For example, a section length of each section (for example, a section length between P8 and P7, a section length between P7 and P6, . . . ) is sequentially subtracted from the returning amount L1. Then, when the remainder becomes less than one section, an internal dividing point corresponding to the remainder is obtained in the target section, so that the reference point coordinate Pa is specified. That is, the reference point coordinate Pa is calculated from a ratio of the remainder to a length of the target section. In the example of FIG. 9, the reference point coordinate Pa is specified between P5 and P4. As a vector facing from the current head coordinate P8 toward the reference point coordinate Pa, a reference vector Va is calculated. The reference vector Va indicates a direction representative of a past tracing result over a relatively large certain range.

Meanwhile, in S16 of FIG. 7, when the moving amount Lx is less than the threshold value Lth, a small returning amount L2 is decided (L1>L2). In this case, as shown in FIG. 10, the reference point coordinate Pa is specified as a point relatively close to the current head coordinate P8, and a relatively short reference vector Va is calculated. The reference point coordinate Pa is set in the same manner as described above. Also in this case, the reference vector Va indicates a direction representative of a past tracing result over a relatively small certain range. The reference point coordinate Pa is a point representative of a past tracing result, and if appropriate as a representative point, the reference point coordinate Pa may be set at a position not on the trace line. For example, the reference point coordinate Pa may be set as a barycentric point of the plurality of trace point coordinates or a point on a regression line defined by a trace line portion extending over a certain range.

In S20 of FIG. 7, an angle θ between the movement vector Vx and the reference vector Va is calculated. A calculation formula for the angle θ can be derived, based on a calculation formula for obtaining an inner product of vectors. In S22, an angle threshold value Δθ is calculated according to a predetermined condition. In the embodiment, one of a large value Δθ1 and a small value Δθ2 is set as the angle threshold value Δθ. Specifically, in a case in which the tracing operation is determined in previous determination, the small angle threshold value Δθ1 is set. On the other hand, in a case in which the deletion operation is determined in the previous determination, when elapsed time since the previous determination is less than a predetermined time, the large angle threshold value Δθ1 is set (kept), and when the elapsed time since the previous determination is equal to or longer than the predetermined time, the angle threshold value Δθ is changed to the small angle threshold value Δθ1. The reason is that when the tracing operation is being performed, the condition for deletion determination is strictly set so that the deletion operation is not to be erroneously determined, and when the deletion operation is being performed, the condition is kept so that the tracing operation is not to be erroneously determined. However, in a case in which the pointer stops over the predetermined time or longer after the deletion operation, there is a high possibility that the tracing operation will be resumed. Therefore, the deletion operation determination condition is set stricter. The order of S20 and S22 may be reversed or these steps may be executed at the same time.

In the meantime, the elapsed time is provided so as to detect a state in which the pointer stops, and corresponds to a duration time of the state in which a change is not detected in S10. This is just an example, and a duration time of a state in which the moving amount or moving speed is equal to or less than a predetermined value may be set as the elapsed time. In a case in which there is no previous determination result, the angle threshold value Δθ1 may be set as a default value.

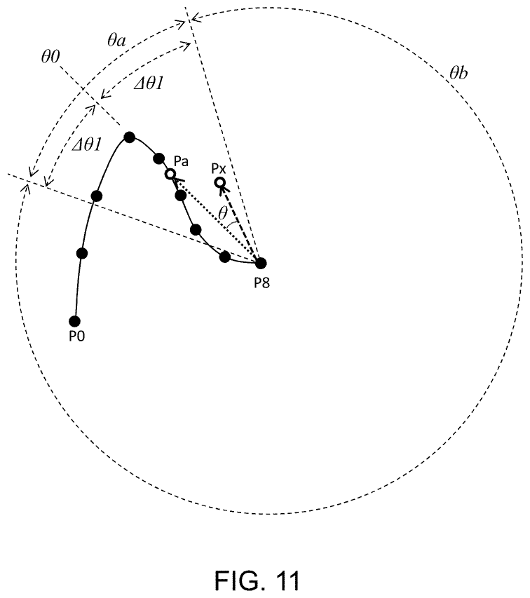

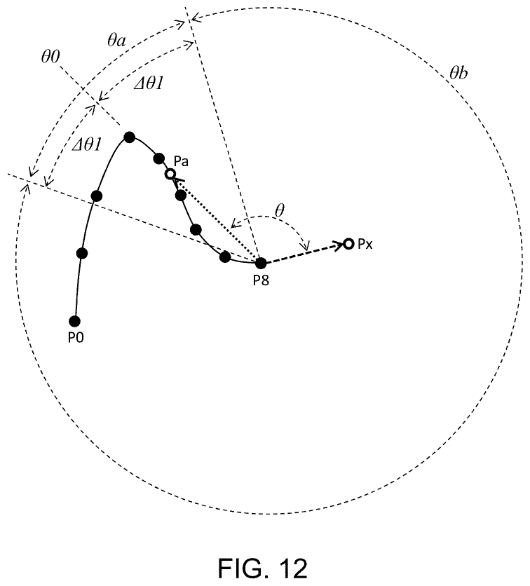

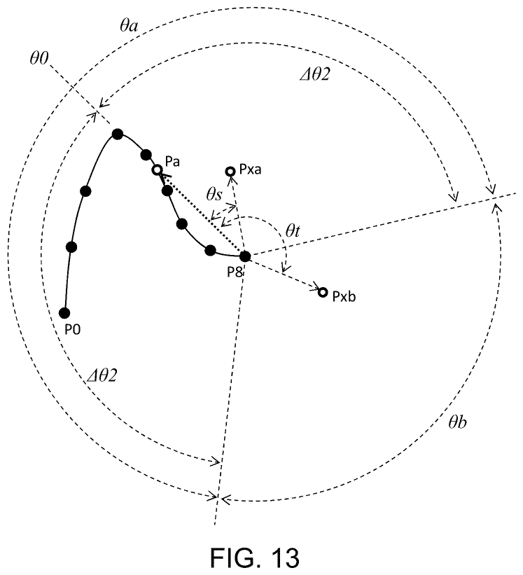

FIG. 11 depicts a set state of the small angle threshold value Δθ1. When the tracing operation is determined in the previous determination, the small angle threshold value Δθ1 is set as the angle threshold value Δθ. Also, when the deletion operation is determined in the previous determination but it is recognized that the pointer has stopped, the small angle threshold value Δθ1 is set as the angle threshold value Δθ. A direction of the reference vector is indicated by θ0. The angle threshold value Δθ1 is set on both sides about the direction, which configures the deletion operation determination condition. When the angle θ between the movement vector and the reference vector is less than the angle threshold value Δθ1, it is determined that this-time operation is the deletion operation, and when the angle θ is equal to or greater than the angle threshold value Δθ1, it is determined that this-time operation is the tracing operation. In the example of FIG. 11, the angle θ is less than the angle threshold value Δθ1. On the other hand, in FIG. 12, the angle θ between the two vectors is equal to or greater than the angle threshold value Δθ1. In this case, it is determined that this-time operation is the tracing operation.

FIG. 13 depicts a set state of the large angle threshold value Δθ2. In this case, when the angle θ between the two vectors is θs, as shown, since the angle θs is less than the angle threshold value Δθ2, it is determined that this-time operation is the deletion operation. That is, the deletion operation determination is repeated. On the other hand, when the angle θ between the two vectors is et, as shown, since the angle et is equal to or greater than the angle threshold value Δθ2, it is determined that this-time operation is the tracing operation. In FIG. 13, the angle threshold value Δθ2 is equal to or greater than 90°. During the fine deletion operation, the pointer may be finely moved. In this case, the large angle threshold value Δθ2 is set so as to prevent erroneous recognition. When the stop state of the pointer occurs, the large angle threshold value is switched to the small angle threshold value Δθ1.

In FIG. 7, in S24, it is determined whether this-time operation (pointer moving operation) is the tracing operation or the deletion operation, according to the aforementioned determination condition. That is, the operation type is determined. When the tracing operation is determined, the processing is shifted from S26 to S28. In S28, the head coordinate of the trace line is updated. That is, the current pointer movement destination coordinate is updated to a new current head coordinate. In S30, the trace line is drawn, based on the trace line coordinate series after the update. At that time, the start point mark and the pointer are also displayed.

On the other hand, in S24, when the deletion operation is determined, the processing is shifted from S26 to S32. In S32, the trace line is partially deleted from the head coordinate of the trace line by a distance equivalent to this-time pointer moving amount. As described below, a new current head coordinate is calculated after the deletion. In S34, the trace line is drawn, based on the trace point coordinate series updated by such processing. At that time, the start point mark and the pointer are also displayed. In S36, it is determined whether or not to continue the processing, and if so, the processing is shifted to S10.

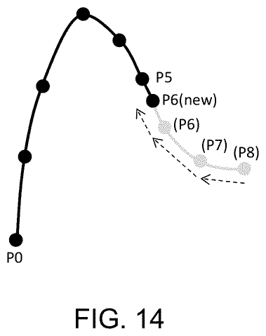

FIG. 14 depicts a specific example of the deletion processing. The trace line is partially deleted from the head thereof by the pointer moving amount. In this case, each section length (for example, a section length between P8 and P7, a section length between P7 and 6, . . . ) is sequentially subtracted from the head coordinate P8. Then, when the remainder as a result of the subtraction becomes less than a target section, an internal dividing point is obtained in the target section, based on the remainder, and the internal dividing point becomes a new current head coordinate. In the example of FIG. 14, P6 (new) is the internal dividing point; i.e., the new current head coordinate. In the example of FIG. 14, each coordinate data of P8, P7 and P6 is deleted from the management table. Then, the coordinate data of P6 (new) is added. The data to be deleted may be temporarily saved in preparation for restoration.

According to the embodiment, when it is intended to delete the trace line, only the pointer moving operation is to be performed. Specifically, when the pointer is moved back to the start point side substantially along the trace line, the mode is automatically switched to the deletion mode. Therefore, it is possible to reduce the user's burden and to improve the operability. Also, according to the embodiment, since the reference point setting condition is switched according to the pointer moving amount, when there is an operation equivalent to the small deletion, the reference point can be set to a point close to the head coordinate, and when there is an operation equivalent to the large deletion, the reference point can be set to a point distant from the head coordinate. That is, since the method of setting the reference direction is switched, it is possible to appropriately set the reference vector in a direction, which represents a portion of interest of the user, according to a size of the portion. According to the embodiment, since the angle threshold value is switched depending on the situation, it is possible to reduce a possibility that the user's unintended processing will be executed arbitrarily. Also, since the size of the portion to be deleted is set according to the pointer moving amount, it is possible to improve the usability.

In the embodiment, when the predetermined knob is subjected to the rotation operation during the execution of the processing shown in FIG. 7, the trace line is partially deleted from the head thereof, according to an angle of the rotation. That is, the user can select the deletion by the pointer moving operation and the deletion by the knob rotating operation.

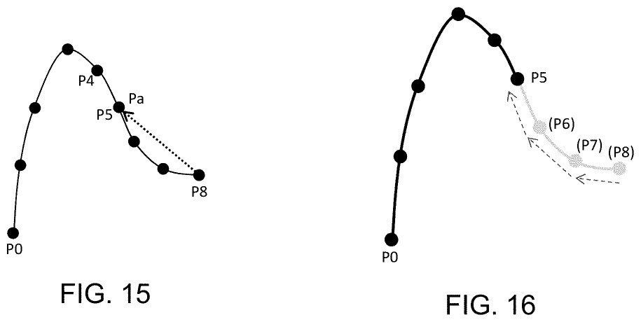

FIG. 15 depicts a modified embodiment of the setting of the reference point coordinate. As shown, as the reference point coordinate Pa, the trace point coordinate may be selected, rather than the internal dividing point coordinate. FIG. 16 depicts a modified embodiment of the deletion processing. As shown, the deletion range may be defined in a section unit. According to the modified embodiments, the calculation on the internal dividing point coordinate can be omitted to reduce an amount of the calculation.

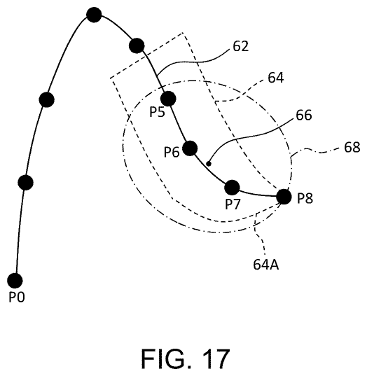

FIG. 17 depicts a modified embodiment of the determination condition. For example, a determination range 64 is set. When this-time pointer movement destination coordinate belongs to the determination range 64, the deletion operation may be determined. The determination range 64 is a band-shaped definite range set along the trace line. The determination range has a spire shape in the vicinity 64A of the head coordinate. A length of the determination range 64 may be fixed but may be changed according to the pointer moving amount.

A barycentric point 66 may be calculated, based on the plurality of trace point coordinates including the head coordinate, and an ellipse having the barycentric point 66 as a center may be defined and set as a determination region 68. When this-time pointer movement destination coordinate belongs to the determination region 68, the deletion operation is determined, and otherwise, the tracing operation is determined. The operation type may be determined, based on both the pointer movement information and the pointer moving amount. In the embodiment, one side and the other side of the reference vector are not distinguished, but they may be distinguished based on the trace point coordinate series. For example, an asymmetrical determination condition may be set.

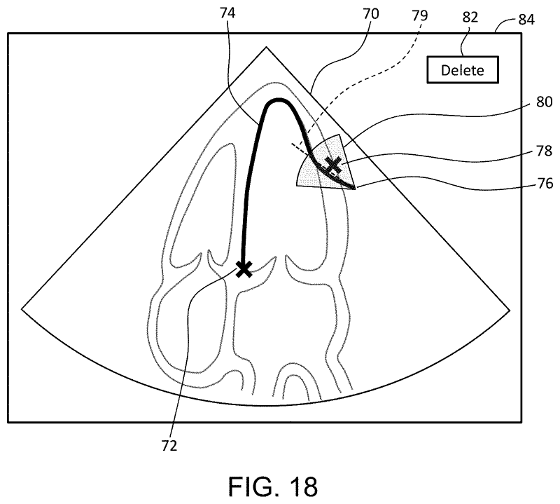

FIG. 18 depicts a first display example. A trace line 74 is displayed on a tomographic image 70, and a start point mark 72 and a pointer 78 are also displayed. When the pointer 78 is moved from the current head coordinate and the movement destination coordinate is decided, or at another timing, for example, a fan-like FIG. 80 indicative of the angle threshold value may be displayed. At that time, a line 79 indicative of the reference vector or a direction thereof may be displayed. Also, an indicator 82, which indicates that the deletion operation is being determined, may be displayed. When the indicator is displayed outside of the tomographic image, it is possible to avoid a situation that the tomographic image 70 or the trace line 74 is hidden by the indicator 82. In the meantime, when displaying the FIG. 80, the figure is preferably displayed in a semi-transparent aspect so that the tomographic image or the trace line is not to be hidden.

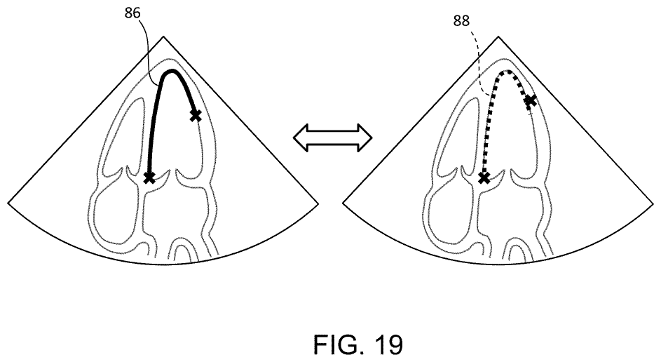

As shown in FIG. 19, a display aspect of the trace line (a color, a thickness, a line type and the like) may be differently set when the tracing operation is determined (refer to reference sign 86) and when the deletion operation is determined (refer to reference sign 88).

The concept of the medical image display device of the present disclosure includes an information processing device configured to process and display a medical image, in addition to the diagnostic device such as the ultrasonograph, the X-ray CT device, and the like. Also, the concept may include a treatment device. The ultrasonic image includes much noise, so that when the manual tracing is performed along the tissue contour, the trace line is likely to deviate from the tissue contour. The above processing is particularly useful for the ultrasonograph.

1. A medical image display device comprising:
a device for drawing a trace line by moving a pointer on a medical image;
a determination unit configured to determine, based on a current trace line extending from a start point coordinate to a current head coordinate and a current pointer coordinate after the current head coordinate is set, whether a pointer moving operation from the current head coordinate to the current pointer coordinate is a tracing operation or a deletion operation; and
a deletion unit that deletes partially or wholly the current trace line when the deletion operation is determined.

2. The medical image display device according to claim 1, wherein the determination unit is configured to set a deletion operation determination condition, based on the current trace line, and
wherein the determination unit determines the deletion operation when the deletion operation determination condition is satisfied by the pointer moving operation.

3. The medical image display device according to claim 2, wherein the determination unit is configured to set an angle condition as the deletion operation determination condition, based on a reference vector from the current head coordinate to a reference point coordinate on the current trace line, and
wherein the determination unit determines the deletion operation when a movement vector from the current head coordinate to the current pointer coordinate satisfies the angle condition.

4. The medical image display device according to claim 2, wherein the determination unit is configured to change the deletion operation determination condition, based on at least one of a previous determination result and a pointer stop time.

5. The medical image display device according to claim 3, wherein the reference point coordinate is a coordinate that is set by tracing back the current trace line from the current head coordinate, and a returning amount at that time is set according to a pointer moving amount from the current head coordinate to the current pointer coordinate.

6. The medical image display device according to claim 1, wherein the deletion unit is configured to set a deletion amount by which the current trace line is to be deleted with being traced back from the current head coordinate, based on a pointer moving amount from the current head coordinate to the current pointer coordinate.

7. The medical image display device according to claim 6, wherein the current trace line is managed as coordinate series composed of a plurality of trace point coordinates, and
wherein when deleting partially the current trace line, some of the coordinate series are invalidated according to the deletion amount, and a new current head coordinate is then added to a head of the remaining coordinate series.

8. The medical image display device according to claim 1, wherein whenever the current pointer coordinate is updated, it is determined whether the pointer moving operation is the deletion operation, and
wherein when the pointer is continuously traced back along the current trace line, the current trace line becomes gradually shorter.

9. The medical image display device according to claim 1, comprising a display for displaying at least one of a determination reference in the determination unit and a determination result of the determination unit.

10. A trace line processing method comprising:
a process of determining, based on a current trace line extending from a start point coordinate to a current head coordinate and a current pointer coordinate after the current head coordinate is set, whether a pointer moving operation from the current head coordinate to the current pointer coordinate is a tracing operation or a deletion operation;
a process of updating the current head coordinate to the current pointer coordinate, when the tracing operation is determined; and
a process of deleting partially or wholly the current trace line when the deletion operation is determined.
